(19) United States
(12) Patent Application Publication
(10) Pub. No.: US 2011/0251443 A1
(43) Pub. Date: Oct. 13, 2011

(54) MANUFACTURE OF VINYL CHLORIDE MONOMER FROM RENEWABLE MATERIALS, VINYL CHLORIDE MONOMER THUS-OBTAINED, AND USE

(75) Inventor: Jean-Luc Dubois, Millery (FR)

(73) Assignee: ARKEMA FRANCE

(21) Appl. No.: 13/131,775

(22) PCT Filed: Nov. 30, 2009
(86) PCT No.: PCT/FR09/52333

§ 371 Date: May 27, 2011

(30) Foreign Application Priority Data
Nov. 28, 2008 (FR) 0858091

(51) Int. Cl. C07C 21/06; C07C 1/00; C07C 17/35; C07C 17/354

(52) U.S. Cl. 570/189; 570/233; 570/217; 570/230

(57) ABSTRACT

The invention relates to a process for the manufacture of vinyl chloride monomer comprising the preparation of acetylene from one or more renewable starting materials and then the reaction of the acetylene with hydrogen chloride in order to form vinyl chloride monomer. The invention also relates to the vinyl chloride monomer obtained and to its use.

The present invention relates to a process for the preparation of vinyl chloride monomer from renewable starting materials and to a vinyl chloride monomer which is obtained, at least in part, from one or more renewable starting materials or which is capable of being obtained by the process.

The vinyl chloride monomer is well known for the use thereof as monomer in (co)polymers. For example, vinyl chloride can be used for the synthesis of polyvinyl chloride.

One of the problems posed by the processes for the synthesis of vinyl chloride monomer of the prior art is that it is carried out starting from non-renewable starting materials of fossil (oil) origin, in particular ethylene. In point of fact, the resources of these starting materials are limited and the extraction of oil requires drilling to increasingly deep depths and under technical conditions which are ever more difficult, requiring sophisticated equipment and the use of processes which are ever more expensive in energy. These constraints have a direct consequence with regard to the cost of manufacturing ethylene and thus with regard to the cost of manufacturing vinyl chloride monomer.

Advantageously and surprisingly, the inventors of the present patent application have employed a process for the industrial manufacture of vinyl chloride monomer from renewable starting materials.

The process according to the invention makes it possible to dispense, at least in part, with starting materials of fossil origin and to replace them with renewable starting materials.

The vinyl chloride monomer obtained according to the process according to the invention is of such a quality that it can be used in all the applications in which the use of vinyl chloride monomer is known.

A subject-matter of the invention is thus a process for the manufacture of vinyl chloride monomer comprising the following stages:

a) preparation of acetylene from one or more renewable starting materials, then

b) reaction of the acetylene with hydrogen chloride to form vinyl chloride monomer.

Another subject-matter of the invention is the vinyl chloride monomer in which at least a portion of the carbon atoms is of renewable origin and the vinyl chloride monomer capable of being obtained by the process according to the invention.

Another subject-matter of the invention is a composition comprising the said vinyl chloride and the use of the said vinyl chloride monomer.

Other subject matters, aspects or characteristics of the invention will become apparent upon reading the following description.

A renewable starting material is a natural resource, for example animal or plant resource, the stock of which can be reconstituted over a short period on the human scale. In particular, it is necessary for the stock to be able to be renewed as quickly as it is consumed. For example, plant materials exhibit the advantage of being able to be cultivated without their consumption resulting in an apparent reduction in natural resources.

Unlike the materials resulting from fossil materials, renewable starting materials comprise ¹⁴C. All the samples of carbon drawn from living organisms (animals or plants) are in fact a mixture of 3 isotopes: ¹²C (representing approximately 98.892%), ¹³C (approximately 1.108%) and ¹⁴C (traces: 1.2×10⁻¹⁰%). The ¹⁴C/¹²C ratio of living tissues is identical to that of the atmosphere. In the environment, ¹⁴C exists in two predominant forms: in the form of carbon dioxide gas (CO₂) and in the organic form, that is to say in the form of carbon incorporated in organic molecules.

In a living organism, the ¹⁴C/¹²C ratio is kept constant metabolically as the carbon is continually exchanged with the external environment. As the proportion of ¹⁴C is constant in the atmosphere, it is the same in the organism, as long as it is living, since it absorbs this ¹⁴C in the same way as the surrounding ¹²C. The mean ¹⁴C/¹²C ratio is equal to 1.2×10⁻¹².

¹²C is stable, that is to say that the number of ¹²C atoms in a given sample is constant over time. ¹⁴C is radioactive and the number of ¹⁴C atoms in a sample decreases over time (t), its half life being equal to 5730 years.

The ¹⁴C content is substantially constant from the extraction of the renewable starting materials up to the manufacture of the vinyl ester according to the invention and even up to the end of the use of the object comprising the vinyl ester.

Consequently, the presence of ¹⁴C in a material, this being the case whatever the amount thereof, gives an indication with regard to the origin of the molecules constituting it, namely that they originate from renewable starting materials and not from fossil materials.

The amount of ¹⁴C in a material can be determined by one of the methods described in Standard ASTM D6866-06 (Standard Test Methods for Determining the Biobased Content of Natural Range Materials Using Radiocarbon and Isotope Ratio Mass Spectrometry Analysis).

This standard comprises three methods for measuring organic carbon resulting from renewable starting materials, known as biobased carbon. The proportions shown for the vinyl choride of the invention are preferably measured according to the mass spectrometry method or the liquid scintillation spectrometry method described in this standard and very preferably by mass spectrometry.

These measurement methods evaluate the ratio of the ¹⁴C/¹²C isotopes in the sample and compare it with a ratio of the ¹⁴C/¹²C isotopes in a material of biological origin giving the 100% standard, in order to measure the percentage of organic carbon in the sample.

Preferably, the vinyl chloride monomer according to the invention comprises an amount of carbon resulting from renewable starting materials of greater than 20% by weight, preferably of greater than 50% by weight, with respect to the total weight of carbon of the vinyl chloride monomer.

In other words, the vinyl chloride can comprise at least 0.25×10⁻¹⁰% by weight of ¹⁴C and preferably at least 0.5×10⁻¹⁰% by weight of ¹⁴C.

Advantageously, the amount of carbon resulting from renewable starting materials is greater than 75% by weight, preferably equal to 100% by weight, with respect to the total weight of carbon of the vinyl chloride monomer.

According to the first embodiment of the invention, the acetylene is prepared according to the following stages:

a) reduction of calcium oxide by carbon resulting from one or more renewable starting materials, in order to form calcium carbide, then

b) hydrolysis of the calcium carbide in order to form acetylene.

The chemical reaction involved during stage a) is as follows:

CaO+3C→CaC₂+CO

The renewable starting material or materials which can be used in the process according to the invention can be chosen from wood charcoal, wood tar, in particular from pine or from straw, heavy residues from the pyrolysis of biomass, in particular of straw, cellulose, straw, wood and lignin.

The wood charcoal can be obtained by any well known conventional method.

Thus, the wood charcoal can be obtained by carbonization according to the following method.

Wooden logs are positioned on the ground in the form of a star, around a post planted in the ground. A cylindrical tank devoid of base and lid is placed on the logs, the post corresponding to the axis of the tank. A floor is constructed on the logs in order to prevent the wood from ending up in contact with the ground and preventing the air and smoke currents from correctly circulating. Around the post, a central chimney is erected with small dry branches and, as the central chimney rises, wood is charged to the space situated between the central chimney and the tank while attempting to create the least space possible. As the tank fills, heavier wood is charged. Once the tank has filled, the post which has acted as guide is removed. Dry twigs, which will be used to start the fire, are positioned in a thin layer at the bottom of the central chimney. At the foot of the tank, between the logs positioned in the form of a star, are inserted, on the one hand, pipes which will make possible the entry of air and, on the other hand, angled pipes which will be used for the exit of smoke and act as side chimneys. The bottom of the central chimney is then set on fire, for example using a lit rag at the end of a stick. When the smoke becomes thick and swirling, the tank is filled as much as possible and then a lid is placed on the tank, the lid exhibiting a central opening acting as chimney. Earth is laid around the tank and over the tank in order to render everything as airtight as possible. It is then possible to attempt to close the opening of the lid. After firing for 10 to 20 h, wood charcoal is obtained in the tank.

The reduction of calcium oxide with carbon to form calcium carbide is generally carried out in a closed furnace equipped with three electrodes.

The closed furnace is generally coated inside with refractory bricks.

The temperature in the furnace is generally between 2200 and 2300° C. The reaction is carried out at atmospheric pressure.

The electrodes can be manufactured in situ with the fines from the renewable starting material or materials. Generally, the electrodes are manufactured from coke. The electrodes are generally gradually introduced into the lime/renewable starting material(s) mixture, bringing about the partial melting and the mutual reaction thereof. The electrodes are generally continuous but can comprise a hollow region which makes possible the injection of fines of starting materials originating from the feeding or from the dust removal, which makes it possible to continuously introduce starting materials directly into the reactor. The electrodes are generally supplied with three-phase alternating current, under a voltage of 100 to 250 V, with a current density of less than 10 A/cm² of electrode surface. The electricity consumption can go up to 3.30 kWh/kg of carbide.

The calcium carbide is obtained in a molten state and is generally run out via orifices made at the base of the furnace. It can be collected in ingot moulds, where it cools off for 1 to 2 h before being removed from the mould in order to be subsequently crushed and sieved.

The production of calcium carbide is accompanied by the release of a large amount of carbon monoxide, generally 400 Sm³/t. This gas includes, on average, 70% by volume of carbon monoxide, and also dust. It can be used as fuel in ancillary plants.

After the stage of reduction of calcium oxide by carbon resulting from one or more renewable materials in order to form calcium carbide, the process according to the invention comprises a stage of hydrolysis of the calcium carbide in order to form acetylene.

The chemical reaction involved is as follows:

C₂Ca+2H₂O→C₂H₂+Ca(OH)₂

This hydrolysis reaction is highly exothermic and calls for strict control of the temperature in order to prevent the acetylene from decomposing.

The hydrolysis stage can be carried out using a wet generator or a dry generator, according to whether the residual lime is extracted in the form of a milk comprising approximately 10% by weight of lime or in the form of hydrated lime without excess water.

Wet generators are used in particular in the production of dissolved acetylene. Among these, devices comprising fall of carbide into water, devices comprising fall of water and contact devices are distinguished.

Dry generators are used in particular in large scale plants. In these dry generators, the water/calcium carbide ratio by weight is generally approximately 1.1.

The hydrolysis of the calcium carbide to form the acetylene generally comprises the stages described below.

The calcium carbide is introduced into a perforated cylinder, for example by means of a screw conveyor. The cylinder is generally present in a concentric casing. The carbide is generally in the form of granules. The reactor is kept stirred in order to prevent the calcium carbide grains from remaining floating at the surface, where they might overheat and ignite the acetylene.

Water is then sprayed into the said cylinder, generally inside the internal calender.

The acetylene formed is then directed from the conveyor to the washing tower and is subjected therein to a further spraying with water. This further spraying with water carries away the greater part of the solids conveyed by the gas. The possible residual lime and the possible impurities of the carbide are generally carried away by a conveyor screw to a vat.

The acetylene is then cooled to a temperature of less than 0° C., preferably of between −5° C. and −15° C., better still of the order of −10° C., in order to condense the greater part of the water.

The acetylene is subsequently purified by contact with sulphuric acid, preferably dilute sulphuric acid, generally in a liquid/liquid absorber. The acetylene is then again purified with sodium hypochlorite, generally prepared by the action of chlorine on sodium hydroxide, in order to remove the impurities.

Generally, this first embodiment of the invention makes it possible to limit the formation of impurities. A high purity acetylene can be obtained by using, as renewable starting material, cellulose, straw, wood or lignin.

In order to limit the impurities, it is also possible to extract them directly from the fresh biomass, rather than, for example, from wood charcoal, which has already greatly changed towards a thermodynamically more stable state.

The acetylene is then cooled, preferably to 0° C., in order to carry out a further separation of the water. The acetylene then still generally includes a small amount of water, less than 0.5% by weight, generally approximately 0.4% by weight. A more thorough dehydration can be obtained by passing over silica gel.

The residual lime can be recycled in the process.

The production of acetylene from charcoal is described in the work Procédés de pétrochimie, Caractéristiques techniques et économiques [Petrochemical Processes, Industrial and Economic Characteristics], 1985, 2^{nd} edition, Volume 1, published by Technip.

According to a second embodiment of the invention, the acetylene is produced from one or more hydrocarbons resulting from one or more renewable starting materials by a process comprising a stage of transfer of energy to the said hydrocarbon(s) and then a stage of quenching.

The production of acetylene from one or more hydrocarbons is based on the thermodynamic properties of the acetylene. Ordinary paraffins and olefins are more stable than acetylene at standard temperatures. When the temperature increases, the free energy of the paraffins and olefins becomes positive, while that of the acetylene decreases. At 1400K, acetylene is the most stable of the ordinary hydrocarbons. However, although it has the lowest free energy of the hydrocarbons at this temperature, acetylene is unstable with regard to its elements C and H₂. As the activation energy for the reaction for the formation of acetylene is greater than that of its decomposition reaction, more acetylene is produced in proportion, the more rapidly the reaction medium is brought to a high temperature. For the same reason, it is necessary for the quenching to be extremely rapid in order to prevent the acetylene from decomposing.

The transfer of energy can be carried out by direct transfer of heat by means of an electric arc or of a plasma, or else by indirect transfer of heat by means of contact bodies or of steam, or else by an autothermal process.

Mention may be made, among electric arc processes, of the Hüls process.

The direct transfer of heat can also be carried out by means of a plasma, generally a thermal plasma, using an arc or high frequency device. In arc plasmas, the ionization of a gas, such as argon or hydrogen, is obtained by passing through an electric arc initiated and maintained between a cathode and an anode. In high frequency plasmas, the ionization of the gas is carried out by passing through a tube, generally made of silica, for example placed in a solenoid through which a high frequency current, generally of between 5 and 60 MHz, passes.

Mention may be made, among plasma processes, of the Hoechst and Hüls processes, which use hydrogen in a device fed with one or more hydrocarbons.

Mention may be made, among the processes having indirect transfer of heat, of the Wulff process and the Kureha process.

In the Wulff process, the operation of the furnace is cyclical: in a first step, the furnace is heated by combustion with air of a fuel (feedstock or other fuel); in a second step, the hydrocarbons to be cracked are decomposed by absorbing the heat accumulated during the preceding period. In practice, the cycle comprises four periods:
a heating phase: the air enters the furnace via one of the ends (right, for example), is heated up through refractory bricks up to a temperature generally of between 980 and 1100° C., and reaches the chamber for injection of the fuels. Combustion brings the temperature generally to 1200-1370° C. The gases discharged via the left part exit at a temperature generally of the order of 315° C. after having reheated the refractory stack;
a cracking phase: the vaporized feedstock enters via the left and flows towards the right as far as the centre, where the vapours are brought to a temperature generally of between 1200 and 1370° C. The cracked gases exit via the right at a temperature generally of the order of 315° C.;
a heating phase, identical to the first, the flow of the fluids being reversed;
a cracking phase, identical to the second, the flow of the fluids being reversed.

The cycle generally lasts one minute.

In the Kureha process, the hydrocarbons are preheated to a temperature generally of the order of 300° C., by heat exchange with combustion flue gases, and are then introduced into a reactor, at the top of which is injected a stream of superheated vapour at 2000° C.

In an autothermal process, the combustion of a portion of the feedstock provides the heat necessary for the cracking reaction of the remainder of the feedstock.

The Hüls, Hoechst, Wulff and Kureha processes and the autothermal processes are described in the work Procédés de pétrochimie—Caractéristiques techniques et économiques, Volume 1, published by Technip.

When the acetylene is produced from one or more hydrocarbons resulting from one or more renewable starting materials by a process comprising a stage of transfer of energy to the said hydrocarbon(s) and then a stage of quenching, the renewable starting material or materials are chosen from biomass pyrolysis tars and biogases.

The production of methane from biomass is known. Thus, methane can be obtained from biogas. Biogas is the gas produced by the fermentation of animal and/or plant organic matter in the absence of oxygen.

This fermentation, also known as methanization, takes place naturally or spontaneously in landfill sites containing organic waste but can be carried out in digesters, in order to treat, for example, sewage sludge, industrial or agricultural organic waste, pig manure or household refuse. Preferably, use is made of biomass containing animal manure which acts as nitrogenous input necessary for the growth of the microorganisms providing the fermentation of the biomass to give methane.

Biogas is composed essentially of methane and carbon dioxide gas. The carbon dioxide gas can be removed by washing the biogas using a basic aqueous sodium hydroxide, potassium hydroxide or amine solution or also with water under pressure or by absorption in a solvent, such as methanol. It is possible to obtain, according to this route, pure methane of uniform quality.

Methanization processes are well known to a person skilled in the art. Reference may in particular be made to the paper Review of Current Status of Anaerobic Digestion Technology for Treatment of Municipal Solid Waste, November 1998, RISE-AT. Mention may also be made of the various existing biological processes for the treatment of wastewater well known to a person skilled in the art, such as the Laran process from Linde.

As explained above, after producing the acetylene, the process according to the invention comprises a stage of reaction of the acetylene with hydrogen chloride in order to form vinyl chloride monomer.

According to a first embodiment of the invention, the reaction of the acetylene with hydrogen chloride is carried out in the presence of a catalyst based on mercury chloride on a support.

According to a second embodiment of the invention, the reaction of the acetylene with hydrogen chloride is carried out in the presence of a liquid catalytic system comprising at least one compound of a metal from Group VIII, a fatty amine hydrochloride, the melting point of which is greater than 25° C., and an organic solvent chosen from aliphatic, cycloaliphatic and aromatic hydrocarbons and their mixtures.

This stage of producing vinyl chloride monomer from acetylene is described in Patent EP 0 525 843.

The term “fatty amine” is understood to mean any amine or mixture of amines comprising a high number of carbon atoms, for example more than 8 carbon atoms, and exhibiting an unbranched or relatively unbranched molecular structure. The preferred amines are those which include from 10 to 20 carbon atoms. Mention may be made, for example, of decylamine, undecylamine, dodecylamine or 3-methyldodecylamine.

Use is preferably made of a catalytic system comprising dodecylamine hydrochloride.

The compounds of metals from Group VIII employed in the catalytic systems of the present invention are generally chosen from iron, cobalt, nickel, ruthenium, rhodium, palladium, osmium, iridium or platinum compounds or their mixtures. The chlorides of these metals from Group VIII are preferred but any other compound which can be converted to the chloride in the presence of hydrogen chloride during the preparation of the catalytic system can also be used.

Preferably, the compound of a metal from Group VIII employed in the present invention is chosen from platinum compounds and palladium compounds, such as platinum(II) chloride or palladium(II) chloride, a platinochloride or a palladochloride of alkali metals or alkaline earth metals, hexachloroplatinic acid or its salts, and palladium compounds in which the palladium has a high valency.

The compounds of metals from Group VIII which are particularly preferred are platinum(II) chloride and palladium(II) chloride. The compound of the metal from Group VIII which is more particularly preferred is palladium(II) chloride.

The choice of the nature of the organic solvent employed in the stage of reaction of acetylene with hydrogen chloride in order to form vinyl chloride monomer is conditioned in particular by the need for it to be inert with regard to the reactants under the reaction conditions, for it to be miscible with the fatty amine hydrochloride at the reaction temperature and for it to be capable of dissolving the latter at a temperature below its melting point. Furthermore, for reasons of safety and of ease of use, preference is given to organic solvents of low volatility. The choice of the organic solvent is also influenced by its ability to absorb acetylene. Solvents which satisfy the various criteria set out above are chosen from aliphatic, cycloaliphatic or aromatic hydrocarbons and their mixtures, for example paraffins having from 7 to 15 carbon atoms and alkylbenzenes, in particular xylenes, propylbenzenes, butylbenzenes and methylbenzenes.

The ratio by weight of the organic solvent to the fatty amine hydrochloride is generally greater than 0.1. Preferably, this ratio is greater than or equal to 0.5. Under particularly preferred conditions, it is greater than or equal to 0.8. Generally, this ratio is less than or equal to 20. Preferably, it is less than or equal to 10. Under particularly preferred conditions, it is less than or equal to 8.

The content of compound of a metal from Group VIII in the catalytic system, expressed in millimoles per litre of solution of catalytic system, is generally greater than or equal to approximately 1 mmol/l, preferably greater than or equal to approximately 10 mmol/l. The content of compound of a metal from Group VIII in the catalytic system is generally less than or equal to approximately 200 mmol/l, preferably less than or equal to approximately 100 mmol/l.

The stage of reaction of the acetylene with hydrogen chloride in order to form vinyl chloride monomer can be carried out from ambient temperature up to 200° C. At a higher temperature, the catalytic system has a tendency to rapidly degrade. Generally, the reaction temperature is such that all the fatty amine hydrochloride is in solution. The preferred reaction temperature, that is to say that offering the best compromise between productive output, yield and stability of the catalytic medium, is greater than or equal to 80° C. The best results are obtained at temperatures of greater than or equal to 120° C. Preferably, the reaction temperature does not exceed 180° C. A reaction temperature of less than or equal to 170° C. is particularly preferred. The process according to the invention is generally carried out at atmospheric pressure or at a slightly greater pressure compatible with the safety regulations for the handling of acetylene, that is to say not exceeding approximately 1.5 bar.

The stage of manufacture of vinyl chloride by hydrochlorination of acetylene in the process according to the invention is carried out by bringing the gaseous reactants, acetylene and hydrogen chloride, into contact with the liquid catalytic system in any appropriate reactor. The process according to the invention can be carried out conventionally in any apparatus which promotes gas/liquid exchange, such as a plate column or a flooded column comprising packing. Another embodiment of the process which makes possible good exchanges of material between the liquid and gas phases consists in employing a counterflow reactor, optionally of the sprayed packing bed type, the liquid catalytic system trickling over the packing, countercurrentwise to the gaseous flow of the reactants.

In the stage of reaction of the acetylene with hydrogen chloride in order to form vinyl chloride monomer of the process according to the invention, the molar ratio of the hydrogen chloride to the acetylene introduced into the reactor is generally greater than or equal to 0.5. Preferably, this ratio is greater than or equal to 0.8. Generally, this molar ratio is less than or equal to 3. Good results have been obtained with a molar ratio of the hydrogen chloride to the acetylene introduced into the reactor of less than or equal to approximately 1.5. The acetylene and the hydrogen chloride can be brought into contact in the reactor or, preferably, mixed prior to the introduction thereof into the reactor.

Advantageously, the process according to the invention can comprise a stage of preparation of vinyl chloride monomer from ethylene obtained from one or more renewable starting materials.

In this case, the preparation of vinyl chloride monomer is generally carried out by conversion of the ethylene to dichloroethane by direct chlorination and then cracking of the dichloroethane in order to form vinyl chloride monomer.

The presence of this stage of preparation of vinyl chloride monomer from ethylene obtained from one or more renewable starting materials makes it possible to provide hydrogen chloride, which can subsequently be used in the reaction for the hydrochlorination of acetylene.

Thus, the reactions are as follows:
hydrochlorination of acetylene:

C₂H₂+HCl→CH₂═CHCl
chlorination of ethylene:

CH₂═CH₂+Cl₂→CH₂Cl—CH₂Cl
cracking of dichloroethane:

CH₂Cl—CH₂Cl→HCl+CH₂═CHCl

or, overall:

C₂H₂+CH₂═CH₂+Cl₂→2 CH₂═CHCl

When the process according to the invention comprises a stage of preparation of vinyl chloride monomer from ethylene obtained from one or more renewable starting materials, the ethylene can be obtained by means of a process comprising a first stage of fermentation of at least one plant material, in order to produce ethanol, and then a second stage of dehydration of the ethanol to give ethylene.

The first stage of the process for producing ethylene obtained from one or more renewable starting materials comprises the fermentation of at least one plant material in order to produce ethanol. This plant material may in particular be chosen from sugars, starch and the plant extracts comprising them, among which may be mentioned beet, sugarcane, cereals, such as wheat, barley, sorghum or maize, and potato, without this list being limiting. It may alternatively be biomass (mixture of cellulose, hemicellulose and lignin). Ethanol is then obtained by fermentation, for example using Saccharomyces cerevisiae. The plant matter employed is generally in the form hydrolyzed before the fermentation stage. This preliminary hydrolysis stage thus makes possible, for example, the saccharification of starch, in order to convert it into glucose, or the conversion of sucrose into glucose.

These fermentation processes are well known to the person skilled in the art. They comprise, for example, the fermentation of plant materials in the presence of one or more yeasts, followed by a distillation which makes it possible to recover the ethanol in the form of a more concentrated aqueous solution, which is subsequently treated for the purpose of further increasing its molar concentration of ethanol.

In the second stage of the process for producing ethylene obtained from one or more renewable starting materials, the ethanol obtained by fermentation is dehydrated in a first reactor to give a mixture of ethylene and water. It is preferable for the alcohol to be injected at the top of the first reactor. This dehydration stage is generally carried out in the presence of a catalyst which can in particular be based on γ-alumina. An example of catalyst suitable for the dehydration of ethanol is sold in particular by Eurosupport under the trade name ESM 110®. It is an undoped trilobe alumina not comprising much residual Na₂O (usually 0.04%). A person skilled in the art will be able to choose the optimum operating conditions for this dehydration stage. By way of example, it has been demonstrated that a ratio of the flow rate by volume of liquid ethanol to the catalyst volume of 1 h⁻¹ and a mean temperature of the catalytic bed of 400° C. results in a virtually complete conversion of the ethanol with a selectivity for ethylene of the order of 98%.

The dehydration can also be carried out in the presence of steam, which then acts also as heat-exchange fluid compensating for the consumption of heat by the dehydration reaction, which is endothermic.

The present invention also relates to a composition comprising the vinyl chloride monomer in which at least a portion of the carbon atoms is of renewable origin, as defined above, or to the vinyl chloride monomer capable of being obtained by the process as defined above.

The present invention also relates to the use of the vinyl chloride monomer according to the invention in the manufacture of polymers, in particular polyvinyl chloride.

The vinyl chloride monomer according to the invention can be converted to PVC by a suspension process. Polyvinyl chloride manufactured by the emulsion or bulk process can also be obtained from the vinyl chloride monomer according to the invention.

1. Process for the manufacture of vinyl chloride monomer comprising the following stages:
a) preparation of acetylene from one or more renewable starting materials, then
b) reaction of the acetylene with hydrogen chloride to form vinyl chloride monomer.

2. Process according to claim 1, characterized in that the acetylene is prepared according to the following stages:
a) reduction of calcium oxide by carbon resulting from one or more renewable starting materials, in order to form calcium carbide, then
b) hydrolysis of the calcium carbide in order to form acetylene.

3. Process according to claim 2, characterized in that the renewable starting material or materials are chosen from wood charcoal, wood tar, in particular from pine or from straw, heavy residues from the pyrolysis of biomass, in particular of straw, cellulose, straw, wood and lignin.

4. Process according to claim 2, characterized in that the reduction of calcium oxide by carbon in order to form calcium carbide is carried out in a closed furnace equipped with three electrodes.

5. Process according to claim 2, characterized in that the hydrolysis of calcium carbide in order to form acetylene comprises the following stages:
calcium carbide is introduced into a perforated cylinder, then
water is sprayed into the said cylinder, then
the acetylene formed is subjected to a further spraying with water in a washing tower, then
the acetylene is cooled to a temperature of less than 0° C., preferably of between −5° C. and −15° C., better still of the order of −10° C., in order to condense most of the water, then
the acetylene is purified by contact with sulphuric acid and then with sodium hypochlorite, then
the acetylene is cooled, preferably to 0° C., in order to carry out a further separation of the water.

6. Process according to claim 1, characterized in that the acetylene is produced from one or more hydrocarbons resulting from one or more renewable starting materials by a process comprising a stage of transfer of energy to the said hydrocarbon(s) and then a stage of quenching.

7. Process according to claim 6, characterized in that the transfer of energy takes place by direct transfer of heat by means of an electric arc or of a plasma, or else by indirect transfer of heat by means of contact bodies or of steam, or else by an autothermal process.

8. Process according to claim 6, characterized in that the renewable starting material or materials are chosen from biomass pyrolysis tars and biogases.

9. Process according to claim 1, characterized in that the reaction of the acetylene with hydrogen chloride is carried out in the presence of a catalyst based on mercury chloride on a support.

10. Process according to claim 1, characterized in that the reaction of the acetylene with hydrogen chloride is carried out in the presence of a liquid catalytic system comprising at least one compound of a metal from Group VIII, a fatty amine hydrochloride, the melting point of which is greater than 25° C., and an organic solvent chosen from aliphatic, cycloaliphatic and aromatic hydrocarbons and their mixtures.

11. Process according to claim 10, characterized in that the fatty amine hydrochloride comprises from 10 to 20 carbon atoms.

12. Process according to claim 10, characterized in that the compound of a metal from Group VIII is chosen from palladium compounds and platinum compounds.

13. Process according to claim 10, characterized in that the ratio by volume of the solvent to the fatty amine hydrochloride varies from 0.1 to 20.

14. Process according to claim 10, characterized in that the content of compound of a metal from Group VIII, expressed in millimoles per litre of the catalytic system, is greater than or equal to 1 mmol/l and less than or equal to 200 mmol/l.

15. Process according to claim 1, characterized in that the reaction of the acetylene with hydrogen chloride is carried out at a temperature of between 80° C. and 180° C.

16. Process according to claim 1, characterized in that the hydrogen chloride and the acetylene are employed in a molar ratio of approximately 0.5 to 3.

17. Process according to claim 1, characterized in that it comprises a stage of preparation of vinyl chloride monomer from ethylene obtained from one or more renewable starting materials.

18. Process according to claim 17, characterized in that the preparation of vinyl chloride monomer is carried out by conversion of the ethylene to dichloroethane by direct chlorination and then cracking of the dichloroethane in order to form vinyl chloride monomer.

19. Vinyl chloride monomer, in which at least a portion of the carbon atoms is of renewable origin.

20. Vinyl chloride monomer, capable of being obtained by the process as defined in claim 1.

21. Vinyl chloride monomer according to claim 19, characterized in that it comprises an amount of carbon resulting from renewable materials of greater than 20%, preferably of greater than 50%, better still of greater than 70%, by weight with respect to the total weight of carbon of the vinyl chloride monomer.

22. Composition comprising the vinyl chloride monomer according to claim 19.

23. Use of the vinyl chloride monomer according to claim 19 in the manufacture of polymers, in particular of polyvinyl chloride.
